(19) United States
(12) Patent Application Publication
(10) Pub. No.: US 2012/0123174 A1
(43) Pub. Date: May 17, 2012

(54) CATALYST FOR SELECTIVE HYDROGENATION OF ACETYLENE COMPOUNDS

(75) Inventors: Moriyasu Sugeta, Toyama (JP); Hirofumi Fukada, Toyama (JP)

(73) Assignee: SUED-CHEMIE CATALYSTS JAPAN, INC., Tokyo (JP)

(21) Appl. No.: 13/121,163

(22) PCT Filed: Sep. 25, 2008
(86) PCT No.: PCT/JP2008/067355

§ 371 Date: Apr. 27, 2011

(51) Int. Cl. C07C 5/09; B01J 27/057; B01J 37/18; B01J 35/02

(52) U.S. Cl. 585/273; 502/100; 502/215

(57) ABSTRACT

The present invention provides a catalyst for selective hydrogenation used to obtain 1,3-butadiene by selectively hydrogenating acetylene compounds contained in a C4 hydrocarbon compound reservoir which is obtained by steam cracking and mainly contains 1,3-butadiene. This catalyst composition mainly contains palladium and bismuth, or palladium, bismuth and tellurium.

### CROSS-REFERENCE TO RELATED APPLICATIONS

The present application is the United States national stage filing under 35 U.S.C. 371 of PCT Application Ser. No. PCT/JP2008/067355, filed on Sep. 25, 2008, the entirety of which is relied upon and incorporated herein by reference in its entirety.

### BACKGROUND OF THE INVENTION

1. Field of the Invention

The present invention relates to a catalyst used in a reaction for obtaining 1,3-butadinene and, more specifically, to a catalyst that selectively hydrogenates acetylene compounds contained in a C4 hydrocarbon compound reservoir which mainly contains 1,3-butadiene.

2. Description of the Related Art

A variety of olefin compounds used as basic raw material in the petrochemical industry are typically produced by steam cracking of naphtha. These olefin compounds contain highly-unsaturated hydrocarbon compounds, and in order to utilize them as a raw material in the macromolecular chemical industry the hydrocarbon compounds must be removed. To produce olefin compounds such as ethylene, propylene, butadiene, isoprene, and other similar compounds, it is necessary to minimize the loss of useful materials in the feed flow, such as the ethylene, propylene, butanes, butadiene, isoprene and other similar compounds, and to remove various acetylene-type impurities such as acetylene, methylacetylene, vinylacetylene, ethylacetylene, 2-methyl-1-butane-3-ene and other similar compounds from the mixed reservoir of unpurified C2 to C5 compounds.

Examples of methods of removing highly-unsaturated hydrocarbon compounds include a method in which these compounds are removed by allowing them to selectively react with hydrogen in the presence of a catalyst and a method of removing these compounds by solvent extraction. When using these methods, it is known that many metals have activity for the hydrogenation reaction of highly-unsaturated hydrocarbon compound. In the case of the hydrogenation reaction carried out in the petrochemical industry to purify olefin compounds, a highly selective catalyst is required to prevent the loss of olefins caused by the hydrogenation of olefin compounds, which is a side reaction. As an appropriate catalyst therefore, a palladium-based catalyst is often used. Such a palladium-based catalyst used in the selective hydrogenation reaction has not only high selectivity, but also excellent activity; therefore, by adjusting the reaction temperature, the catalyst is used to inhibit side reactions and un-reacted highly-unsaturated hydrocarbon compounds. Yet, when comparing the reactivity for olefin compounds and acetylene compounds, the palladium-based catalyst has markedly higher reactivity for acetylene compounds.

However, since the amount of olefin compounds normally existing in the reaction is much greater than that of acetylene compounds, hydrogenation of olefin compounds also occurs along with hydrogenation of acetylene compounds. From the standpoint of the yield of a desired compound, it is preferable that the catalyst allow a minimal amount of the hydrogenation reaction of the olefin compounds. Further, as another side reaction, precipitation of carbonaceous substances also occurs on the catalyst due to polymerization of highly-unsaturated compounds. It is, therefore, also preferred that such precipitation of polymerized substances be minimized as much as possible since it can reduce the regeneration rate of the catalyst.

It is especially difficult to remove trace amounts of C4 acetylene compounds such as vinylacetylene and ethylacetylene from a C4 hydrocarbon reservoir mainly containing 1,3-butadiene by hydrogenation, and butadiene is also hydrogenated to generate butane. However, since butadiene is useful as a polymer raw material and the loss thereof leads to a large economical loss, it is preferable to minimize such loss as much as possible.

Previously, as a method of improving the performance of a palladium-based catalyst, it is reported in U.S. Pat. No. 4,547,600 that selectivity is improved by adding Ag to the catalyst composition. In addition, U.S. Pat. No. 6,459,008 reports that the selectivity is improve by adding Ag and Bi to Pd in the catalyst composition. Further, Japanese Patent Publication No. S62-23726 reports a catalyst in which Te or Sb was added in order to suppress the elution of Pd into highly-concentrated butadiene flow. However, while the suppression of Pd elution is described therein, there is no description regarding improvement of the catalyst selectivity. That is, none of the prior arts could completely remove C4 acetylenes while maintaining the activity of the palladium-based catalyst for selective hydrogenation of C4 acetylene compounds at a high level and also in terms of the selectivity level, without a loss of 1,3-butadiene caused by excessive hydrogenation.

It has previously been reported that palladium/alumina catalysts result in improved selectivity through the an addition of a co-catalyst component; however, they have practical problems which could not be satisfied by the prior art. Accordingly, there is a continued need for a catalyst that selectively hydrogenate acetylene compounds contained in a C4 hydrocarbon compound reservoir containing mostly 1,3-butadiene.

To the extent that specific publications are discussed above in this Description of the Related Art Section, these discussions should not be taken as an admission that the discussed publications (for example, published patents) are prior art for patent law purposes. For example, some or all of the discussed publications may not be sufficiently early in time, may not reflect subject matter developed early enough in time and/or may not be sufficiently enabling so as to amount to prior art for patent law purposes. To the extent that specific publications are discussed above in this Description of the Related Art Section, they are all hereby incorporated by reference into this document in their respective entireties.

### BRIEF SUMMARY OF THE INVENTION

It is therefore a principal object and advantage of the present invention to provide a catalyst that selectively hydrogenate acetylene compounds contained in a C4 hydrocarbon compound reservoir which contains mostly 1,3-butadiene.

It is another object and advantage of the present invention to provide a method of use of a catalyst that selectively hydrogenate acetylene compounds contained in a C4 hydrocarbon compound reservoir which contains mostly 1,3-butadiene.

Other objects and advantages of the present invention will in part be obvious, and in part appear hereinafter.

In accordance with the foregoing objects and advantages, the present invention provides a catalyst for selectively hydrogenating an acetylene compound in a C4 hydrocarbon compound reservoir comprising 1,3-butadiene. The catalyst comprises a first metal compound supported by an inorganic carrier, and a second metal compound, wherein the first metal compound is a palladium compound and the second metal compound is a metal compound other than palladium, and further wherein the catalyst is reduced in an air flow that comprises hydrogen. In a preferred embodiment, the second metal compound comprises a bismuth compound, or a bismuth compound and a tellurium compound. The first metal compound is preferably present in the catalyst at about 0.01 to 1 by wt %, and the second metal compound is present at about 0.01 to 10 wt % based on the total weight of the catalyst. The weight ratio of the second metal compound to the palladium compound is preferably about 0.1 to 10. The tellurium compound is present at about 0.01 to 0.1 wt % based on the total weight of the catalyst. Additionally, the catalyst is reduced at a temperature of about 250 to 600° C. Further, the C4 hydrocarbon compound reservoir comprises 1,3-butadiene at an amount of about 30 to 60% and the acetylene compound at an amount of about 0.1 to 5%.

According to a second aspect of the invention is provided a method for producing the catalyst described herein. The method comprises the steps: (i) contacting a palladium compound supported by an inorganic carrier with a reducing agent to produce a first intermediate; (ii) drying or calcinating the first intermediate; (iii) adding a metal compound to the dried or calcinated first intermediate to produce a second intermediate; (iv) drying or calcinating the second intermediate; and reducing the dried or calcinated second intermediate in an air flow at high temperature, where the air flow comprises hydrogen.

According to a third aspect of the invention is provided a method for using the catalyst described herein to selectively hydrogenate acetylene compounds. The method comprises the step of contacting the catalyst with a C4 hydrocarbon compound reservoir containing 1,3-butadiene.

### BRIEF DESCRIPTION OF THE SEVERAL VIEWS OF THE DRAWING(S)

The present invention will be more fully understood and appreciated by reading the following Detailed Description in conjunction with the accompanying drawings, in which:

FIG. 1 depicts a physical property of a catalyst according to one embodiment of the present invention as measured by an X-ray diffraction (“XRD”) measuring apparatus; and

FIG. 1(b) depicts a physical property of the catalyst according to one embodiment of the present invention as measured by an XRD measuring apparatus.

### DETAILED DESCRIPTION OF THE INVENTION

In order to solve the aforementioned problems, the inventors intensively studied to discover a catalyst which hardly hydrogenates 1,3-butadiene, that is, a highly selective catalyst that can be produced by adding a novel co-catalyst component to a palladium catalyst and reducing it with hydrogen at a high temperature. Therefore, described below is a catalyst that hydrogenates acetylene compounds in 1,3-butadiene. The catalyst is characterized by containing palladium and bismuth, or palladium, bismuth, and tellurium and performing the hydrogenation at a high temperature. Also described below is a method of producing the catalyst and a method of using the catalyst.

By using the catalyst according to the present invention, selective hydrogenation reaction of acetylene compounds such as vinylacetylene and ethylacetylene which are contained at a trace amount in a C4 hydrocarbon compound reservoir mainly containing 1,3-butadiene can be carried out without losing 1,3-butadiene due to hydrogenation; therefore, a catalyst having much higher selectivity compared to conventional catalysts, a method of producing the catalyst, and a method of using the catalyst can be provided.

The catalyst can be prepared by allowing an inorganic carrier to support a palladium compound; treating the inorganic carrier with a reducing agent, followed by drying or calcinating in the air; introducing thereto a metal compound; drying or calcinating the resultant; and subsequently reducing it at a high temperature in an air flow containing hydrogen.

The palladium raw material of the catalyst composition is not particularly restricted as long as it does not contain a component which is degraded by a heat treatment and eventually remains on the catalyst to be a catalyst poison. As such palladium raw material, inorganic and organic compounds, such as palladium chloride, palladium nitrate, tetrachloropalladium, or acetonato palladium may be employed, and the amount of palladium supported on the carrier is preferably in the range of 0.01 to 1% by weight based on the total weight of the catalyst composition.

As the carrier, metal oxides such as alumina, silica, titania, or silica-alumina can be used, and alumina is particularly preferred. Alumina may be used in the form of a molded product such as a tableted product, cylindrical product, extruded product or spherical product, and alumina having a surface area in the range of 1 to 350 m²/g may be used.

Palladium is supported onto the carrier by, for example, a soaking method in which a support solution containing palladium is prepared and the carrier is soaked there into, a spray method in which a support solution is sprayed to the carrier, or an impregnation method in which a support solution is prepared at an amount comparable to the amount of water absorption by the carrier and the carrier is impregnated with the entire amount of the support solution. After being made to support palladium, the carrier is reduced after drying or calcination thereof, or alternatively without drying. The reduction is carried out with hydrogen or by liquid-phase reduction. In the liquid-phase reduction, an aqueous solution of a reducing agent such as formalin, sodium formate or hydrogenated boron can be used, and after the reduction the carrier is washed with water and dried or calcinated. Here, the temperature of the calcination or reduction is not particularly restricted; however, it is in the range of room temperature to 600° C. in a preferred embodiment.

It is desired that palladium be supported on the carrier in an eggshell structure. When the thickness of the palladium layer from the outer surface of the catalyst is not greater than 400 μm, the loss of butadiene is small and a high selectivity can be attained.

The metal compound used as co-catalyst component of the present invention contains bismuth and may be used in the form of a nitrate salt thereof, an inorganic salt thereof such as a chloride salt or an organic salt thereof such as an acetic acid salt. The content of the bismuth compound is preferably in the range of approximately 0.01 to 10% by weight, more preferably in the range of 0.1 to 6% by weight, based on the total weight of the catalyst composition. The weight ratio of the bismuth compound to the palladium compound is preferably 0.1 to 10, more preferably 0.5 to 8.

As the co-catalyst component according to the present invention, a tellurium compound may be further added to the bismuth compound. The tellurium compound may be used in the form of an oxoacid such as a telluric acid salt, an inorganic salt such as a chloride salt, or an oxide. The content of the tellurium compound is preferably in the range of approximately 0.01 to 0.1% by weight, more preferably at an amount of approximately 0.01 to 0.05% by weight, based on the total weight of the catalyst composition.

The co-catalyst component is supported onto the carrier in the same manner as in the case of palladium, for example, by a soaking method, spray method or impregnation method. The aforementioned metal compounds may be supported onto the carrier simultaneously with palladium, or the co-catalyst component may also be further added to the carrier already supporting palladium.

After being made to support these metal catalyst components, the carrier is dried or calcinated, and then reduced in hydrogen gas flow. Here, the calcination temperature is not particularly restricted; however, it is preferably in the range of room temperature to 800° C.

The gas used in the reduction may also contain an inert gas such as nitrogen in addition to hydrogen. The reduction may be carried out at a temperature in the range of 250 to 600° C., and it is preferably carried out at a temperature in the range of 300 to 500° C. This reduction carried out in hydrogen gas flow at a high temperature is critical, and at approximately 150° C., which is a temperature normally used for reduction, the selectivity is reduced. In addition, even with an addition of silver to the catalyst component, a practically sufficient selectivity cannot be attained by hydrogen reduction at a low temperature. The reason why a high selectivity is attained by the reduction at a high temperature is believed to be the generation of compounds such as PdBi and PdBi₂ due to alloying of Pd and the co-catalyst component caused by the reduction at a high temperature.

As a result of the analyses on the catalyst physical properties by X-ray diffraction (“XRD”) analysis, when the catalyst according to the present invention containing Pd and the co-catalyst component was reduced at a high temperature, there was a novel peak which was not observed when the catalyst was reduced at a low temperature; therefore, it is believed that an alloy of Pd and the co-catalyst component, Bi, was generated by the reduction at a high temperature. Since this XRD measurement is based on the crystalline regularity, information regarding the crystal such as strain and size can be obtained in addition to the crystal quality. The presence of a different peak indicates a change in the crystal structure even in the same composition. For example, generation of an alloy such as PdBi₂ results in a peak at such a position indicating the aforementioned change. In this manner, the catalyst according to the present invention is intrinsically different from those known catalysts in that an active component of alloyed metal is generated by carrying out reduction of the catalyst at a high temperature in hydrogen gas flow. This novel catalyst having such physical property selectively hydrogenates trace amounts of C4 acetylenes such as vinylacetylene and ethylacetylene that are contained in a C4 hydrocarbon compound reservoir containing a large amount of 1,3-butadiene.

For the selective hydrogenation reaction using the catalyst according to the present invention, either a fixed-bed-type reactor or fluidized-bed-type reactor may be used, and the reaction may also be carried out in any of a gas phase, liquid phase, or mixed phase. In the gas phase reaction, for example, the ratio of the olefin compounds existing at the reactor outlet can be increased by feeding via the reactor inlet using a carrier gas a mixture of an acetylene compound and an olefin compound such as 1,3-butadiene, as well as hydrogen gas, into the reactor to which the catalyst according to the present invention has already been loaded.

The C4 hydrocarbon compound reservoir mainly containing 1,3-butadiene in which the catalyst according to the present invention is used refers to a compound reservoir which contains butane, isobutane, butene, isobutene, 1,3-butadiene, 1,2-butadiene, ethylacetylene, vinylacetylene, or small amounts of other C3 and C5 hydrocarbons, and it mainly contains 1,3-butadiene at an amount of 30 to 60% and vinylacetylene and ethylacetylene at an amount of 0.1 to 5%.

Further, the selective hydrogenation reaction in which the catalyst according to the present invention is used is optimally carried out under the conditions of: the reaction temperature at 40 to 100° C.; the pressure at 0.5 to 5 MPa; LHSV=1 to 20 h-1; and hydrogen/acetylenes=0.5 to 2, in a preferred embodiment of the present invention. Other ranges may be used as further described herein or would be obvious to one of ordinary skill in the art.

The method according to the present invention will now be described by way of representative examples thereof; however, the present invention is not restricted thereto.

### Example 1

Added to 1,200 ml of pure water was 18.3 ml of palladium chloride solution (120 g/L), and 1,000 g of alumina carrier was further added thereto. The mixture was left to stand for 90 minutes and after removing the solution, the resultant was reduced with 5% Na formate solution at 60° C., washed with water, and then dried in the air to obtain a Pd catalyst. On another front, 4.3 g of bismuth nitrate was completely dissolved in a solution obtained by adding 15 ml of concentrated nitric acid to 165 ml of pure water. The solution was added to the Pd catalyst (300 g), and the resultant was calcinated at 500° C. and subsequently reduced in hydrogen at 350° C. to obtain a catalyst. This catalyst had a composition ratio of Pd/Bi=0.2%/0.6%.

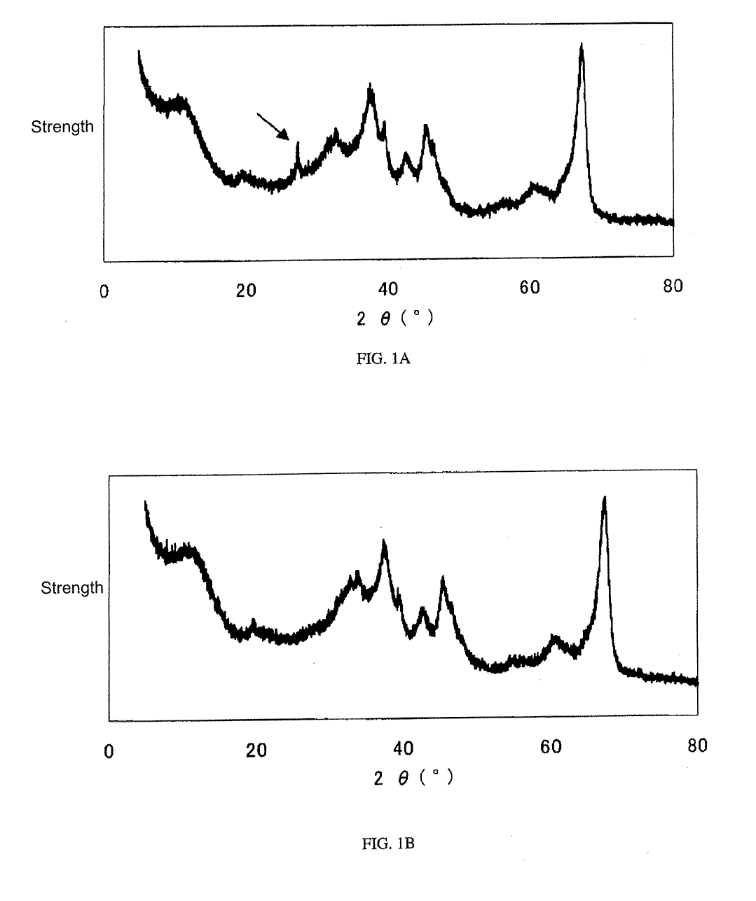

This catalyst was subjected to a measurement by an XRD measuring apparatus. The measurement was carried out by using a copper anticathode at an output of 40 mA and 45 kV and a wavelength of 1.54 angstrom. The result is shown in FIG. 1(a). There was a peak at about 2θ=28°, which was not observed in FIG. 1(b) showing peaks for the later-described catalyst according to Comparative Example 4, below.

### Example 2

A Pd catalyst was obtained in the same manner as in Example 1. On another front, 2.9 g of bismuth nitrate was completely dissolved in a solution obtained by adding 10 ml of concentrated nitric acid to 170 ml of pure water. The solution was added to the Pd catalyst (300 g), and the resultant was calcinated at 500° C. and subsequently reduced in hydrogen at 350° C. to obtain a catalyst. This catalyst had a composition ratio of Pd/Bi=0.2%/0.4%.

### Example 3

A Pd catalyst was obtained in the same manner as in Example 1. On another front, 5.6 g of bismuth nitrate was completely dissolved in a solution obtained by adding 20 ml of concentrated nitric acid to 160 ml of pure water. The solution was added to the Pd catalyst (300 g), and the resultant was calcinated at 500° C. and subsequently reduced in hydrogen at 350° C. to obtain a catalyst. This catalyst had a composition ratio of Pd/Bi=0.2%/0.8%.

### Example 4

To 200 g of sodium chloride aqueous solution (1.2% by weight), 3.7 g of palladium chloride powder was dissolved and pure water was added to obtain 1,200 g of palladium aqueous solution. After making the temperature of this solution 60° C., 1,000 g of alumina carrier was added and the resulting solution was left to stand for 90 minutes. After removing the solution, the resultant was reduced with 5% Na formate solution at 60° C., washed with water and then dried in the air to obtain a Pd catalyst. On another front, 10 g of bismuth nitrate was completely dissolved in a solution obtained by adding 35 ml of concentrated nitric acid to 145 ml of pure water. The solution was added to the Pd catalyst (300 g), and the resultant was calcinated at 500° C. and reduced in hydrogen at 350° C. to obtain a catalyst. This catalyst had a composition ratio of Pd/Bi=0.2%/1.4%.

### Example 5

Added to 1,200 ml of pure water was 18.3 ml of palladium chloride solution (120 g/L), and 1,000 g of alumina carrier was further added thereto. The mixture was left to stand for 90 minutes and after removing the solution, the resultant was put into 1,000 ml of 5% Na formate solution at 60° C., left to stand for 3 hours, washed with water and then dried in the air to obtain a Pd catalyst. On another front, 4.3 g of bismuth nitrate and 0.162 g of telluric acid were completely dissolved in a solution obtained by adding 15 ml of concentrated nitric acid to 165 ml of pure water. The solution was added to the Pd catalyst (300 g), and the resultant was calcinated at 500° C. and reduced in hydrogen at 350° C. to obtain a catalyst. This catalyst had a composition ratio of Pd/Bi/Te=0.2%/0.6%/0.02%.

### Comparative Example 1

Added to 1,200 ml of pure water was 18.3 ml of palladium chloride solution (120 g/L), and 1,000 g of alumina carrier was further added thereto. The mixture was left to stand for 90 minutes and after removing the solution, the resultant was put into 1,000 ml of 5% Na formate solution at 60° C., left to stand for 3 hours, washed with water and then dried in the air to obtain a Pd catalyst. On another front, 0.47 g of silver nitrate was completely dissolved in 180 ml of pure water, and after adding the solution to the Pd catalyst (300 g), the resultant was calcinated at 500° C. to obtain a catalyst. This catalyst had a composition ratio of Pd/Ag=0.2%/0.1%.

### Comparative Example 2

Added to 1,200 ml of pure water was 18.3 ml of palladium chloride solution (120 g/L), and 1,000 g of alumina carrier was further added thereto. The mixture was left to stand for 90 minutes and after removing the solution, the resultant was put into 1,000 ml of 5% Na formate solution at 60° C., left to stand for 3 hours, washed with water and then dried in the air to obtain a Pd catalyst. On another front, 4.3 g of bismuth nitrate was completely dissolved in a solution obtained by adding 15 ml of concentrated nitric acid to 165 ml of pure water. The solution was added to the Pd catalyst (300 g) and the resultant was calcinated at 500° C. to obtain a PD/Bi catalyst. Further, after completely dissolving 0.14 g of silver nitrate in 180 ml of pure water, the solution was added to the Pd/Bi catalyst (300 g), and the resultant was calcinated at 500° C. to obtain a catalyst. This catalyst had a composition ratio of Pd/Bi/Ag=0.2%/0.6%/0.03%.

### Comparative Example 3

Added to 1,200 ml of pure water was 18.3 ml of palladium chloride solution (120 g/L), and 1,000 g of alumina carrier was further added thereto. The mixture was left to stand for 90 minutes and after removing the solution, the resultant was put into 1,000 ml of 5% Na formate solution at 60° C., left to stand for 3 hours, washed with water and then dried in the air to obtain a Pd catalyst. On another front, after completely dissolving 0.16 g of telluric acid in 180 ml of pure water, the solution was added to the Pd catalyst (300 g), and the resultant was calcinated at 500° C. to obtain a catalyst. This catalyst had a composition ratio of Pd/Te=0.2%/0.03%.

### Comparative Example 4

A catalyst was prepared in the same manner as in Example 1 except for the reduction temperature. The reduction was carried out with hydrogen at 150° C. This catalyst had a composition ratio of Pd/Bi=0.2%/0.6%.

This catalyst was subjected to a measurement by an XRD measuring apparatus. The measurement was carried out by using a copper anticathode at an output of 40 mA and 45 kV and a wavelength of 1.54 angstrom. The result is shown in FIG. 1(b). As clearly seen from the figure, there was no peak observed at about 2θ=28°, which was observed for the catalyst according to Example 1.

### Test Example

Using the catalysts according to Examples 1 and 2 and Comparative Examples 1 to 4, their performances of selectively hydrogenating acetylene compounds were evaluated under the following reaction conditions:

Amount of catalyst=30 ml

LHSV=10 h-1

Pressure=2.9 MPa

Reaction temperature=35° C.

Hydrogen/acetylene=1.0

Recycle ratio=0.5 to 1.0

The reaction ratio is a ratio at which the post-reaction solution is mixed with the raw material to dilute the raw material. Recycle ratio=Amount of post-reaction solution (ml/h)/amount of raw material solution (ml/h). Composition of raw material:

Vinylacetylene=0.3 to 0.5%

Ethylacetylene=0.13 to 1.16%

1,3-butadiene=44%

1-butene=18%

t-2-butene=5%

c-2-butene=4%

i-butene=24%

N-butane=4%

Measurement Method: The degree of acetylene conversion was determined by measuring the concentrations of vinylacetylene and ethylacetylene in the solution before and after the reaction by gas chromatography and using the following equation.

Degree of acetylene conversion={(Δ vinylacetylene+Δ ethylacetylene)/(inlet vinylacetylene+inlet ethylacetylene)}×100(%) FORMULA 1

Δ vinylacetylene=inlet vinylacetylene−outlet vinylacetylene

Δ ethylacetylene=inlet ethylacetylene−outlet ethylacetylene

The ratio of butadiene loss was determined by measuring the concentration of 1,3-butadiene in the raw material and the post-reaction solution and using the following equation.

Butadiene loss=(Δ1,3-butadiene/inlet 1,3-butadiene)×100(%) FORMULA 2

Δ1,3-butadiene=inlet 1,3-butadiene−outlet 1,3-butadiene

The test results are shown in Table 1. When compared at the same degree of C4 acetylene conversion, the catalysts according to Examples 1 and 2 resulted in a smaller 1,3-butadiene loss than the catalysts according to Comparative Examples 1 to 4, and the catalysts according to Examples had a higher selectivity.

TABLE 1

| | Recycle = 0 | | Recycle = 0.5 | | Recycle = 1.0 | |
|---|---|---|---|---|---|---|
| | Degree of C4 | Ratio of | Degree of C4 | Ratio of | Degree of C4 | Ratio of |
| | acetylene | butadiene | acetylene | butadiene | acetylene | butadiene |
| | conversion (%) | loss (%) | conversion (%) | loss (%) | conversion (%) | loss (%) |
| Example 1 | 60.4 | −0.7 | 72.0 | 0.2 | 83.8 | 2.2 |
| Example 2 | 55.4 | −1.2 | 67.9 | −0.9 | 77.5 | 0.9 |
| Example 3 | 56.2 | −1.1 | 75.1 | −0.6 | 82.7 | 0.5 |
| Example 4 | 64.0 | −0.6 | 77.2 | 0.5 | 82.5 | 2.2 |
| Example 5 | 51.8 | −1.0 | 70.3 | 0.5 | 79.6 | 3.4 |
| Comparative | 32.0 | −0.3 | 50.5 | 2.3 | 59.3 | 4.1 |
| Example 1 | | | | | | |
| Comparative | 53.2 | −0.1 | 64.0 | 0.9 | 72.1 | 2.7 |
| Example 2 | | | | | | |
| Comparative | 48.1 | 1.3 | 60.3 | 2.4 | 74.7 | 4.0 |
| Example 3 | | | | | | |
| Comparative | 41.8 | −1.1 | 57.1 | 0.4 | 66.0 | 1.2 |
| Example 4 | | | | | | |

The catalyst for selective hydrogenation according to the present invention can selectively hydrogenate trace amounts of acetylene hydrocarbons contained in a hydrocarbon reservoir obtained by steam cracking or the like, particularly in a C4 hydrocarbon compound reservoir from which separation of acetylene hydrocarbons was conventionally very difficult since a large amount of 1,3-butadiene is contained. At the same time, the catalyst for selective hydrogenation according to the present invention can prevent excessive hydrogenation reaction. Therefore, it minimizes the loss of useful resources and is very advantageous in industrial cracking processes. Further, the catalyst according to the present invention can drastically reduce the cost for producing a final raw material for macromolecular chemical industry.

Although the present invention has been described in connection with a preferred embodiment, it should be understood that modifications, alterations, and additions can be made to the invention without departing from the scope of the invention as defined by the claims.

What is claimed is:

1. A catalyst for selectively hydrogenating an acetylene compound in a C4 hydrocarbon compound reservoir comprising 1,3-butadiene, said catalyst comprising a first metal compound supported by an inorganic carrier, and a second metal compound, wherein said first metal compound is a palladium compound and said second metal compound is a metal compound other than palladium, and further wherein said catalyst is reduced in an air flow, said air flow comprising hydrogen.

2. The catalyst of claim 1, wherein said second metal compound comprises a bismuth compound.

3. The catalyst of claim 1, wherein said second metal compound comprises a bismuth compound and a tellurium compound.

4. The catalyst of claim 1, wherein said first metal compound is present in said catalyst at about 0.01 to 1 by wt %, and said second metal compound is present at about 0.01 to 10 wt % based on the total weight of said catalyst.

5. The catalyst of claim 4, wherein the weight ratio of said second metal compound to said palladium compound is about 0.1 to 10.

6. The catalyst of claim 3, wherein said tellurium compound is present at about 0.01 to 0.1 wt % based on the total weight of said catalyst.

7. The catalyst of claim 1, wherein said catalyst is reduced at a temperature of about 250 to 600° C.

8. The catalyst of claim 1, wherein said C4 hydrocarbon compound reservoir comprises said 1,3-butadiene at an amount of about 30 to 60% and said acetylene compound at an amount of about 0.1 to 5%.

9. A method for producing the catalyst according to claim 1, comprising the steps:
contacting a palladium compound supported by an inorganic carrier with a reducing agent to produce a first intermediate;
drying or calcinating said first intermediate;
adding a metal compound to said dried or calcinated first intermediate to produce a second intermediate;
drying or calcinating said second intermediate; and
reducing said dried or calcinated second intermediate in an air flow at high temperature, said air flow comprising hydrogen.

10. A method for using the catalyst of claim 1, comprising the step of contacting said catalyst with a C4 hydrocarbon compound reservoir containing 1,3-butadiene.
